(19) United States
(12) Patent Application Publication
(10) Pub. No.: US 2001/0053350 A1
(43) Pub. Date: Dec. 20, 2001

(54) Cosmetic composition comprising N-ethyloxycarbonyl-4-amino-phenol and arbutin or its derivatives and/or ellagic acid or its derivatives

(75) Inventors: Veronique Chevalier, Villecresnes (FR); Dang-Man Pham, Sucy-En-Brie (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'OREAL, Paris (FR)

(21) Appl. No.: 09/739,874

(22) Filed: Dec. 20, 2000

(30) Foreign Application Priority Data
Dec. 20, 1999 (FR) 9916074

(51) Int. Cl. A61K 7/135; A61K 7/021; A61K 31/7048; A61K 31/7034

(52) U.S. Cl. 424/63; 424/401; 514/25; 514/27

(57) ABSTRACT

A depigmenting and/or lightening composition is provided, wherein the composition contains N-ethyloxycarbonyl-4-para-aminophenol and at least one depigmenting agent selected from arbutin and its derivatives and/or ellagic acid and its derivatives, and its use in a method for depigmenting and/or lightening skin.

### BACKGROUND OF THE INVENTION

1. Field of the Invention

The present invention relates to a composition comprising, in a physiologically acceptable medium, N-ethyloxycarbonyl-4-para-aminophenol and at least one depigmenting agent selected from arbutin and its derivatives and/or ellagic acid and its derivatives.

2. Discussion of the Background

For a number of years, considerable resources have been employed with a view to providing topical depigmenting substances which are innocuous but highly effective.

These substances are sought after in particular for the purpose of treating local hyperpigmentation due to melanocytic hyperactivity such as the idiopathic melasmas which arise during pregnancy (“pregnancy mask” or chloasma) or during oestrone/progestagen contraception, localized hyperpigmentations due to benign melanocytic proliferation and hyperactivity, such as senile pigmentary blemishes known as actinic lenigines, accidental hyperpigmentations or depigmentations, possibly as a result of photosensitization or post-lesional scarring, and also certain leucodermias, such as vitiligo. In the latter cases (the instances of scarring which may result in a scar giving the skin a paler appearance, and leucoderinias), where it is not possible to repigment the damaged skin, the remaining normal skin regions are depigmented so as to give the whole of the skin a homogeneous lighter coloring.

Various depigmenting agents have thus been proposed in the prior art.

In particular, the applicant has demonstrated that certain aminophenol derivatives possess the property of inhibiting melanogenesis, even at low concentrations, without exhibiting cytotoxicity. These compounds, and their preparation process, are described in WO 99/10318.

Other well-known depigmenting agents comprise hydroquinone and its derivatives such as arbutin and its esters; ascorbic acid and its derivatives, especially glycosylated derivatives; kojic acid and its esters; ellagic acid and its derivatives; plant extracts, especially extracts of liquorice, of mulberry or of skullcap; and glutathione, cysteine and precursors thereof.

However, more powerful and effective depigmenting agents are desired, which are still physiologically compatible and toxicologically safe.

### SUMMARY OF THE INVENTION

Accordingly one object of the present invention is to provide a depigmenting agent that overcomes the deficiencies of prior art depigmenting agents and provides a significantly more powerful and effective depigmenting effect relative to known compositions.

This and other objects of the present invention have been satisfied by the discovery of a depigmenting composition comprising N-ethyloxycarbonyl-4-para-aminophenol and a compound selected from the group consisting of arbutin, arbutin derivatives, ellagic acid, ellagic acid derivatives and mixtures thereof. This composition has been found to have a depigmenting power which is greater on a molar basis than that of these compounds taken alone. In other words, this combination potentializes the efficacy of these compounds, which produce a synergistic effect in the lightening or depigmentation of the skin or the hair of the body or scalp.

### DETAILED DESCRIPTION OF THE PREFERRED EMBODIMENTS

The present invention relates to a composition comprising, in a physiologically acceptable medium, N-ethyloxycarbonyl-4-para-aminophenol and at least one depigmenting agent selected from arbutin and its derivatives and/or ellagic acid and its derivatives.

Within the context of the present invention, the term “physiologically acceptable medium” means a medium appropriate for topical application to the skin or its exoskeletal appendages, i.e. is compatible with the skin, hair of the body or scalp, nails and mucosae.

Arbutin is a glucoside of hydroquinone whose formula is

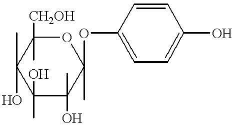

Suitable arbutin derivatives for use in the present compositions can be any derivative that retains its depigmenting capability. Preferably the derivatives are the esters of arbutin, such as the arbutin monoesters described in the applications EP-0 597 776 and EP-0 895 779, which are incorporated here by reference, and the warbutoside esters described in the application EP-0 524 109, whose content is likewise incorporated here by reference.

Ellagic acid and its derivatives comprise the compounds of formula (I)

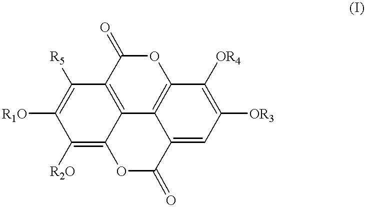

in which

R₁ to R₄ are each, independently, hydrogen atoms, an alkyl group having 1 to 20 carbon atoms (preferably 1 to 10 carbon atoms, more preferably 1-6 carbon atoms), an alkoxy group having 1 to 20 carbon atoms (preferably 1 to 10 carbon atoms, more preferably 1-6 carbon atoms), an alkoxy group having 1 to 20 carbon atoms (preferably 1 to 10 carbon atoms, more preferably 1-6 carbon atoms), a poly(ethylene oxide) or poly(propylene oxide) or poly(ethylene/propylene oxide) (a copolymer of ethylene oxide and propylene oxide units) radical or a sugar radical, and

R₅ is a hydrogen atom, a hydroxyl group or an alkoxy group having 1 to 18 carbon atoms (preferably 1 to 10 carbon atoms, more preferably 1-6 carbon atoms).

These compounds are described in the patent U.S. Pat. No. 5,073,545, and their method of preparation is disclosed in the application JP-53-14605, each of which are hereby incorporated by reference.

The present invention also provides for the use of this composition in a cosmetic preparation for depigmenting and/or whitening the human skin or the hair of the body or scalp.

It further provides for the use of this composition for the manufacture of a preparation for depigmenting and/or whitening the human skin or the hair of the body or scalp.

The present invention likewise relates to a cosmetic method of depigmenting and/or lightening the human skin or the hair of the body or scalp, which consists in applying to the skin or the hair of the body or scalp a composition of the invention.

In the composition of the invention, N-ethyloxycarbonyl-4-para-aminophenol and ellagic acid (or derivative thereof) and/or arbutin (or derivative thereof) are present in an amount such that they act synergistically to confer on the composition a depigmenting effect greater than that obtained with a composition containing only one of these compounds. For example, N-ethyloxycarbonyl-4-para-aminophenol is preferably present in an amount ranging from 0.01 to 10% by weight, and more preferably from 0.5 to 3% by weight, relative to the total weight of the composition. For its part, arbutin or derivative thereof preferably represents from 0.01 to 5% by weight, more preferably from 0.1 to 2% by weight, relative to the total weight of the composition. Finally, ellagic acid or derivative thereof preferably represents from 0.001 to 10% by weight, and more preferably from 0.005 to 3% by weight, relative to the total weight of the composition.

The composition of the invention may be present in any of the pharmaceutical forms which are normally used for topical application. Preferred forms of the composition include, but are not limited to, aqueous, aqueous-alcoholic or oily solutions, oil-in-water or water-in-oil or multiple emulsions, aqueous or oily gels, liquid, pastelike or solid anhydrous products, oil dispersed in an aqueous phase with the aid of spherules, it being possible for these spherules to be polymeric nanocapsules or more preferably, lipid vesicles of ionic and/or nonionic type.

While the aminophenol derivative and ellagic acid employed in the present invention may give rise to solubility problems in certain cosmetic compositions, it is possible to remedy these problems, in accordance with one preferred embodiment, by dissolving the N-ethyloxycarbonyl-4-para-aminophenol in a solubilizer selected from an ester of a fatty acid and an oxyalkylenated polyol, an oxyalkylenated fatty alcohol and mixtures thereof before introducing it into the aqueous phase or into the fatty phase of the composition of the invention. Moreover, it may be advantageous to dissolve the ellagic acid in a basic solution with a pH greater than 14, then to acidify this solution to a pH of between approximately 6 and 8 prior to its incorporation into the composition of the invention.

The present composition may be more or less fluid and may have the appearance of a white or colored cream, a pomade, a milk, a lotion, a serum, a paste or a mousse. The composition can also be applied in aerosol form to the skin or hair of the body or scalp. Additionally, the present composition can be present in solid form and, for example, in stick form. It may be used as a care product and/or as a makeup product for the skin. It may also be in the form of shampoo or conditioner.

Conventionally, the composition of the invention may also comprise one or more adjuvants which are customary in the cosmetic field, such as hydrophilic or lipophilic gelling agents, hydrophilic or lipophilic active principles, preservatives, antioxidants, solvents, fragrances, fillers, filters, pigments, odor absorbers and colorants. The amounts of these various adjuvants are those which are conventionally used in the field in question, and, for example, are from 0.01 to 20% of the total weight of the composition. These adjuvants, depending on their nature, may be introduced into the fatty phase, into the aqueous phase, into the lipid vesicles and/or into the nanoparticles. In any case, these adjuvants and their proportions will be selected so as not to detract from the desired properties of the combination of depigmenting agents according to the invention. Such adjuvant selection is well within the skill of one of ordinary skill in the art.

When the composition of the invention is an emulsion, the proportion of the fatty phase preferably ranges from 5 to 80% by weight, and more preferably from 5 to 50% by weight, relative to the total weight of the composition. The oils, emulsifiers and coemulsifiers used in the composition in emulsion form are selected from those which are conventionally used. The emulsifier and coemulsifier are present in the composition in a proportion preferably ranging from 0.3 to 30% by weight, and more preferably from 0.5 to 20% by weight, relative to the total weight of the composition.

Suitable preferred oils which may be used in the invention, include mineral oils (liquid petroleum), oils of vegetable origin (avocado oil, soya oil), oils of animal origin (lanolin), synthetic oils (perhydrosqualene), silicone oils (cyclomethicone) and fluorinated oils (perfluoropolyethers). Fatty alcohols (cetyl alcohol), fatty acids, and waxes (camauba wax, ozokerite) may also be used as fatty substances.

Suitable preferred emulsifiers and coemulsifiers which may be used in the invention include fatty acid polyethylene glycol esters such as PEG-20 stearate and fatty acid glycerol esters such as glyceryl stearate.

Suitable preferred hydrophilic gelling agents, include carboxyvinyl polymers (carbomer), acrylic copolymers such as acrylate/alkyl acrylate copolymers, polyacrylamides, polysaccharides, natural gums and clays, and, as lipophilic gelling agents, mention may be made of modified clays such as bentones, metal salts of fatty acids, hydrophobic silica and polyethylenes.

As active principles, the composition of the invention preferably comprises at least one UV filter and/or at least one keratolytic agent and/or exfoliator.

The depigmenting or lightening composition of the present invention is applied in accordance with conventional regimens of depigmentation treatment.

### EXAMPLES

Having generally described this invention, a further understanding can be obtained by reference to certain specific examples which are provided herein for purposes of illustration only and are not intended to be limiting unless otherwise specified.

### Example 1

Demonstration of the Synergistic Effect of the Compounds of the Invention

A biological test in vitro (inhibition of the activity of fungal tyrosinase) demonstrated the depigmenting activity of the combinations of compounds according to the invention.

This test consisted in measuring the absorbance (optical density OD) at 475 nm, as a function of time, for each of the following active depigmenting principles:

N-ethyloxycarbonyl-4-para-aminophenol arbutin in solution (at different concentrations) in an appropriate solvent in the presence of a phosphate buffer, 1 ml of L-tyrosine at 2.0×10⁻³ M in a phosphate buffer, 0.1 ml of L-dopa at 1.0×10⁻⁴ M in a phosphate buffer and 50 μl of 1 g/l fungal tyrosinase solution in a phosphate buffer, compared with a control consisting of the solvent used, at the same concentration in the phosphate buffer. Percentage inhibition was subsequently determined by the following equation:

$$on = \frac{{OD}_{max , 475 nm} ( control ) - {OD}_{max , 475 nm} ( active princip}{{OD}_{max , 475 nm} ( control )}$$

For each of the three active compounds above, the molar concentration at which an inhibition figure of 25% was observed was subsequently calculated. This gave a concentration of C₁ of N-ethyloxycarbonyl-4-para-aminophenol and a concentration C₂ of arbutin where:

C₁=1.25×10⁻⁴ M (solvent:ethanol)

C₂=3.33×10⁻⁴ M (solvent:water)

For its part, ellagic acid was used at its upper solubility limit in the solvent (DMSO), i.e. at a concentration C₃=1.35×10⁻⁴ M. At this concentration, ellagic acid has no inhibitory effect on tyrosinase activity.

Subsequently, N-ethyloxycarbonyl-4-para-aminophenol and arbutin were mixed in concentrations C₁ and C₂ and N-ethyloxycarbonyl-4-para-aminophenol and ellagic acid were mixed in respective concentrations C₁ and C₃. Subsequently, the theoretical percentage inhibition of the mixture (or IC theoretical) was calculated, corresponding to the sum of the percentage inhibitions observed for the individual compounds, and the percentage inhibition obtained (or IC obtained) was determined.

The results are as follows:

Mixture C₁+C₂:

IC theoretical=47.3%

IC obtained=58.0%

Mixture C₁+C₃:

IC theoretical=24.8%

IC obtained=59.0%

Taking into account the precision of this test (of the order of 5%), the synergistic effect of the two compounds in combination was thus clearly demonstrated.

### Example 2 (Comparative)

The test described in example 1 was reproduced except that N-ethyloxycarbonyl-4-para-aminophenol was replaced by N-cholesteryloxycarbonyl-4-para-aminophenol.

The results obtained were as follows:

C′₁=6.67×10⁻⁵M

C₂=3.33×10⁻⁴ M

Mixture C′₁+C₂:

IC theoretical=22.5%

IC obtained=23.2%

It is clear that, taking into account the fact that the relative error of the measurement is 5%, no synergistic effect was observed for the combination of this aminophenol derivative with arbutin.

### Example 3 (Comparative)

The depigmenting effect of a mixture of N-ethyloxycarbonyl-4-para-aminophenol and hydroquinone, which is a well-known depigmenting agent whose chemical structure is similar to that of arbutin (arbutin being a glucoside of hydroquinone), was determined in the same way as in Example 1.

A 25% inhibition of the tyrosinase activity was observed for a concentration C₄ of hydroquinone where C₄=8×10⁻⁵ M. The theoretical percentage inhibition was 49.2% for the mixture of the two compounds in concentrations C₁ and C₄. The percentage inhibition obtained was 29.4%.

Therefore, no synergistic effect was demonstrated.

### Example 4

O/W Emulsion with Oleosomes

The following composition was prepared as described in the application EP-A-705 593. The amounts given below are weight percentages.

| | |
|---|---|
| N-Ethyloxycarbonyl-4-para-aminophenol | 0.5% |
| Arbutin | 1.0% |
| Stearyl heptanoate and stearyl octanoate | 5.5% |
| Vegetable oils | 11.6% |
| Preservatives | 0.29% |
| UVA filter | 1.9% |
| Triethanolamine | 0.75% |
| Disodium EDTA | 0.05% |
| Cyclopentasiloxane | 3.7% |
| Glycerol | 3% |
| Sucrose tristearate | 2% |
| oxyethylenated sorbitan monostearate (4 EO) | 1.35% |
| Stearic acid | 1% |
| Oxyethylenated hydrogenated castor oil (60 EO) | 2.5% |
| Tocopheryl acetate | 0.5% |
| Water qs | 100% |

This gives a whitening cream for the face.
Example 5: Depigmenting gel
The following composition is prepared conventionally:

| | |
|---|---|
| N-Ethyloxycarbonyl-4-para-aminophenyl | 0.5% |
| Ellagic acid | 0.2% |
| Preservatives | 0.65% |
| UVB filter | 0.5% |
| Disodium EDTA | 0.1% |
| Sodium hydroxide | 0.11% |
| Carbomers | 0.4% |
| Cyclohexasiloxane | 5% |
| Glycerol | 10% |
| Ethanol | 2.5% |
| Water qs | 100% |

This gives a gel highly suited to reducing actinic lentigines on the hands.

### Example 6

O/W Emulsion without Surfactant

The following composition is prepared conventionally:

| | |
|---|---|
| N-Ethyloxycarbonyl-4-para-aminophenol | 0.5% |
| Arbutin | 2% |
| Vegetable oils | 12% |
| Preservatives | 0.3% |
| UVA filter | 2% |
| UVB filter | 4% |
| Disodium EDTA | 0.05% |
| Diglycol/CHDM/isophthalates/SIP copolymer | 2% |
| (Eastman AQ 55S from EASTMAN CHEMICAL) | |
| Cyclopentasiloxane | 6% |
| Glycerol | 5% |
| Ethanol | 10% |
| Water | qs 100% |

This gives a whitening cream which is particularly suitable for sensitive or reactive skins.

This application is based on French Patent Application No. 9916074, filed with the French Patent Office on Dec. 20, 1999, the entire contents of which are hereby incorporated by reference.

Obviously, additional modifications and variations of the present invention are possible in light of the above teachings. It is therefore to be understood that within the scope of the appended claims, the invention may be practiced otherwise than as specifically described herein.

1. A depigmenting composition comprising:
N-ethyloxycarbonyl-4-para-aminophenol and a depigmenting compound selected from the group consisting of arbutin, arbutin derivatives, ellagic acid, ellagic acid derivatives and mixtures thereof.

2. The depigmenting composition as claimed in claim 1, further comprising a physiologically acceptable medium.

3. The depigmenting composition as claimed in claim 1, wherein the N-ethyloxycarbonyl-4-para-aminophenol is present in an amount of from 0.01 to 10% by weight relative to total weight of the composition.

4. The depigmenting composition as claimed in claim 3, wherein the N-ethyloxycarbonyl-4-para-aminophenol is present in an amount of from 0.5 to 3% by weight relative to total weight of the composition.

5. The depigmenting composition as claimed in claim 1, wherein said depigmenting compound is arbutin or an arbutin derivative and is present in an amount of from 0.01 to 5% by weight relative to total weight of the composition.

6. The depigmenting composition as claimed in claim 5, wherein said depigmenting compound is arbutin or an arbutin derivative and is present in an amount of from 0.1 to 2% by weight relative to total weight of the composition.

7. The depigmenting composition as claimed in claim 1, wherein said depigmenting compound is ellagic acid or an ellagic acid derivative and is present in an amount of from 0.001 to 5% by weight relative to total weight of the composition.

8. The depigmenting composition as claimed in claim 7, wherein said depigmenting compound is ellagic acid or an ellagic acid derivative and is present in an amount of from 0.005 to 3% by weight relative to total weight of the composition.

9. The depigmenting composition as claimed in claim 1, further comprising a member selected from the group consisting of UV filtering compounds, keratolytic agents, exfoliators and mixtures thereof.

10. A cosmetic preparation for depigmenting human skin or hair, comprising:
N-ethyloxycarbonyl-4-para-aminophenol and a depigmenting compound selected from the group consisting of arbutin, arbutin derivatives, ellagic acid, ellagic acid derivatives and mixtures thereof, in a cosmetic carrier.

11. The cosmetic preparation as claimed in claim 10, wherein the N-ethyloxycarbonyl-4-para-aminophenol is present in an amount of from 0.01 to 10% by weight relative to total weight of the preparation.

12. The cosmetic preparation as claimed in claim 11, wherein the N-ethyloxycarbonyl-4-para-aminophenol is present in an amount of from 0.5 to 3% by weight relative to total weight of the preparation.

13. The cosmetic preparation as claimed in claim 10, wherein said depigmenting compound is arbutin or an arbutin derivative and is present in an amount of from 0.01 to 5% by weight relative to total weight of the preparation.

14. The cosmetic preparation as claimed in claim 13, wherein said depigmenting compound is arbutin or an arbutin derivative and is present in an amount of from 0.1 to 2% by weight relative to total weight of the preparation.

15. The cosmetic preparation as claimed in claim 10, wherein said depigmenting compound is ellagic acid or an ellagic acid derivative and is present in an amount of from 0.001 to 5% by weight relative to total weight of the preparation.

16. The cosmetic preparation as claimed in claim 15, wherein said depigmenting compound is ellagic acid or an ellagic acid derivative and is present in an amount of from 0.005 to 3% by weight relative to total weight of the preparation.

17. The cosmetic preparation as claimed in claim 10, further comprising a member selected from the group consisting of UV filtering compounds, keratolytic agents, exfoliators and mixtures thereof.

18. The cosmetic preparation as claimed in claim 10, wherein said preparation is a cream, a pomade, a milk, a lotion, a serum, a paste, a mousse, an aerosol, an emulsion, a solid, a solution or a gel.

19. A method of depigmenting or lightening the skin, hair or both, comprising:
applying to an area of skin, hair or both in need thereof, a depigmenting composition comprising N-ethyloxycarbonyl-4-para-aminophenol and a depigmenting compound selected from the group consisting of arbutin, arbutin derivatives, ellagic acid, ellagic acid derivatives and mixtures thereof.

20. The method of claim 19, wherein said depigmenting composition further comprises a physiologically acceptable medium.

21. The method of claim 19, wherein the N-ethyloxycarbonyl-4-para-aminophenol is present in an amount of from 0.01 to 10% by weight relative to total weight of the composition.

22. The method of claim 21, wherein the N-ethyloxycarbonyl-4-para-aminophenol is present in an amount of from 0.5 to 3% by weight relative to total weight of the composition.

23. The method of claim 19, wherein said depigmenting compound is arbutin or an arbutin derivative and is present in an amount of from 0.01 to 5% by weight relative to total weight of the composition.

24. The method of claim 23, wherein said depigmenting compound is arbutin or an arbutin derivative and is present in an amount of from 0.1 to 2% by weight relative to total weight of the composition.

25. The method of claim 19, wherein said depigmenting compound is ellagic acid or an ellagic acid derivative and is present in an amount of from 0.001 to 5% by weight relative to total weight of the composition.

26. The method of claim 25, wherein said depigmenting compound is ellagic acid or an ellagic acid derivative and is present in an amount of from 0.005 to 3% by weight relative to total weight of the composition.

27. The method of claim 19, wherein said composition further comprises a member selected from the group consisting of UV filtering compounds, keratolytic agents, exfoliators and mixtures thereof.
